(19) United States
(12) Patent Application Publication
(10) Pub. No.: US 2009/0117645 A1
(43) Pub. Date: May 7, 2009

(54) Biochips and methods of fabricating the same

(75) Inventors: Won-Sun Kim, Gyeonggi-do (KR); Jung-Hwan Hah, Gyeonggi-do (KR); Sung-Min Chi, Gyeonggi-do (KR); Sun-Ok Jung, Gyeonggi-do (KR); Man-Hyoung Ryoo, Gyeonggi-do (KR)

Correspondence Address: MILLS & ONELLO LLP, ELEVEN BEACON STREET, SUITE 605, BOSTON, MA, 02108, US

(73) Assignee: Samsung Electronics Co., Ltd., Suwon-si (KR)

(21) Appl. No.: 12/229,645

(22) Filed: Aug. 26, 2008

(30) Foreign Application Priority Data
Aug. 27, 2007 (KR) 10-2007-0086255

(51) Int. Cl. C12M 1/34; B05D 5/00

(52) U.S. Cl. 435/287.2; 427/256

(57) ABSTRACT

Biochips for analyzing components of a biological sample using probes and methods of fabricating the same are provided. In some embodiments, a biochip includes a substrate, a plurality of probes immobilized on a top surface of the substrate, and a capping layer formed on a bottom surface of the substrate.

### CROSS-REFERENCE TO RELATED APPLICATION

This application claims priority from Korean Patent Application No. 10-2007-0086255 filed on Aug. 27, 2007, in the Korean Intellectual Property Office, the disclosure of which is incorporated herein by reference in its entirety.

### FIELD OF THE INVENTION

1. Field of the Invention

The present invention relates to biochips and methods of fabricating the same, for example, to biochips for analyzing components of a biological sample using probes, and methods of fabricating the same.

2. Background

Biochips exemplified by microarrays can analyze selected components of biological samples by exposing the biological samples to probes immobilized on a substrate and observing reactions that occur between the probes and the biological samples. A wide variety of different kinds of probes can be immobilized on a biochip, so a large amount of data can be read in one cycle of an experiment. Today, rapidly advancing high integration technology has allowed vast amounts of data to be collected.

However, an increase in the amount of collected data does not necessarily mean an increase in the reliability of the analysis of the biological samples. Rather, from a certain standpoint, the high integration technology can accelerate generation of data noise that can degrade the reliability of the data. The data noise may become severe when the probes undesirably become coupled to a bottom surface of the substrate.

In addition, from the standpoint of analysis efficiency, in embodiments in which a transparent substrate is used for a microarray, light emitted from a fluorescent material is allowed to pass through the substrate, but only approximately half of the light transmitted during fluorescence detection is utilized.

### SUMMARY OF THE INVENTION

Embodiments of the present invention provide biochips having improved analysis reliability and/or increased analysis efficiency.

Embodiments of the present invention also provide methods of fabricating a biochip having improved analysis reliability and/or increased analysis efficiency.

According to an aspect of the present invention, there is provided a biochip including a substrate, a plurality of probes immobilized on a top surface of the substrate, and a capping layer on a bottom surface of the substrate.

According to another aspect of the present invention, there is provided a method for fabricating a biochip including forming a capping layer on a bottom surface of a substrate, and immobilizing a plurality of probes on a top surface of the substrate.

The above and other aspects and features of the present invention will be described in or be apparent from the following description of the embodiments.

### BRIEF DESCRIPTION OF THE DRAWINGS

The above and other features and advantages of the present invention will become more apparent by describing in detail embodiments thereof with reference to the attached drawings in which:

FIG. 1 is a layout view of biochips according to some embodiments of the present invention;

FIG. 2 is a sectional view of a biochip according to an embodiment of the present invention;

FIG. 3 is a conceptual diagram of a fluorescence analysis in some embodiments of the present invention;

FIGS. 4 and 5 are sectional views of biochips according to other embodiments of the present invention; and

FIGS. 6 through 9 are sectional views illustrating a method of fabricating biochips according to some embodiments of the present invention.

### DETAILED DESCRIPTION OF THE INVENTION

Advantages and features of the present invention and methods of accomplishing the same may be understood more readily by reference to the following detailed description of embodiments and the accompanying drawings. The present invention may, however, be embodied in many different forms and should not be construed as being limited to the embodiments set forth herein. Rather, these embodiments are provided so that this disclosure will be thorough and complete to those skilled in the art, and the present invention will only be defined by the appended claims. Accordingly, in some specific embodiments, well known materials or methods have not been described in detail.

It is noted that the use of any and all examples, or exemplary terms provided herein is intended merely to better illuminate the description and is not a limitation on the scope of the invention unless otherwise specified. The use of the terms “a” and “an” and “the” and similar terms in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

The embodiments will be described with reference to perspective views, cross-sectional views, and/or plan views, in which embodiments of the invention are shown. Thus, the profile of an exemplary view may be modified according to manufacturing techniques and/or allowances. That is, the embodiments of the invention are not intended to limit the scope of the present invention but cover all changes and modifications that can be caused due to a change in manufacturing process. In the drawings, various components are exaggerated or reduced for clarity. Like reference numerals refer to like elements throughout the specification.

Biochips according to some embodiments of the present invention analyze biomolecules contained in biological samples and are used, for example, in gene expression profiling, genotyping through detection of mutation or polymorphism such as Single-Nucleotide Polymorphism (SNP), a protein or peptide assay, potential drug screening, development and preparation of novel drugs, etc. Biochips employ appropriate probes according to the kind of biological sample to be analyzed. Examples of probes useful for biosensors include a DNA probe, a protein probe such as an antibody/antigen or a bacteriorhodopsin, a bacterial probe, a neuron probe, and so on. A biosensor fabricated in the form of a chip may also be referred to as a biochip. For example, depending on the kind of probe used, the biosensor may be referred to as a DNA chip, a protein chip, a cellular chip, a neuron chip, and so on.

Biochips according to some embodiments of the present invention may comprise oligomer probes, suggesting that the number of monomers contained in the oligomer probe is on the level of oligomers. The oligomer can have a molecular weight of about 1,000 or less but the present invention is not limited thereto. The oligomer may include about 2-500 monomers, for example, about 5-30 monomers. However, the characteristics of an oligomer probe are not limited to the ranges listed above. The monomers that are included in an oligomer probe may be nucleosides, nucleotides, amino acids, peptides, etc., according to the type of biological sample to be analyzed.

As used herein, the terms “nucleosides” and “nucleotides” include not only known purine and pyrimidine bases, but also methylated purines or pyrimidines, acylated purines or pyrimidines, etc. Furthermore, the “nucleosides” and “nucleotides” include not only known (deoxy)ribose, but also modified sugars that contain a substitution of a halogen atom or an aliphatic group for at least one hydroxyl group or is functionalized with ether, amine, or the like.

As used herein, the term “amino acids” refers to not only naturally occurring, L-, D-, and nonchiral amino acids, but also to modified amino acids, amino acid analogs, etc.

As used herein, the term “peptides” refers to compounds produced by an amide bond between a carboxyl group of one amino acid and an amino group of another amino acid.

Unless otherwise specified in the following exemplary embodiments, the term “probe” is a DNA probe, which is an oligomer probe including (e.g., consisting of) about 5-30 covalently bound monomers. However, the present invention is not limited to the probes listed above and a variety of probes may used.

Embodiment of the present invention will now be described with reference to the accompanying drawings.

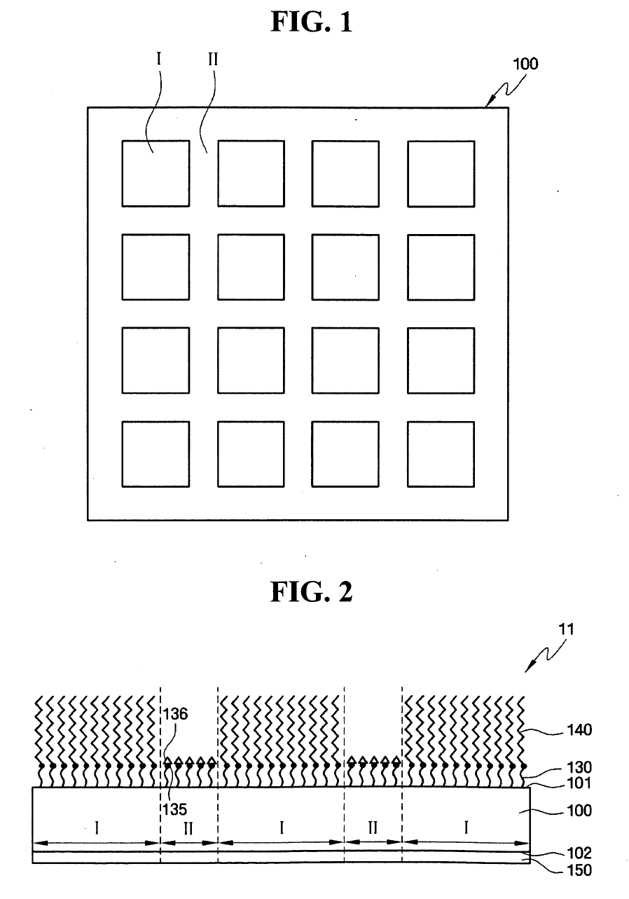

FIG. 1 is a layout view of biochips according to some embodiments of the present invention.

Referring to FIG. 1, a substrate 100 for a biochip according to an exemplary embodiment of the present invention includes a plurality of probe cell regions I and non-probe cell regions II. The probe cell regions I and the non-probe cell regions II are defined according to the presence or absence of immobilized probes 140, respectively. In other words, the probe cell region I of the substrate 100 is a region of the substrate 100 on which a plurality of plurality of probes 140 is immobilized, while the non-probe cell region II is a region of the substrate 100 on which the probes 140 are not immobilized. Probe cells including immobilized plurality of plurality of probes 140 are formed on the probe cell region I of the substrate 100.

In some embodiments, the probes 140 of the same sequences are immobilized on a probe cell region I, and in other embodiments, probes 140 of different sequences may be immobilized on different probe cell regions I

Different probe cell regions I are separated from each other by the non-probe cell region II. Thus, as shown, each probe cell region I is surrounded by the non-probe cell region II. The plurality of probe cell regions I may be arranged in a matrix configuration. The matrix configuration does not necessarily have a regular pattern.

Unlike the separated probe cell regions I, the non-probe cell regions II may be connected to one another into a single unit. For example, the non-probe cell regions II can be arranged in a lattice configuration.

FIG. 2 is a sectional view of a biochip according to an embodiment of the present invention.

Referring to FIG. 2, the biochip 11 according to an embodiment of the present invention includes a substrate 100, a plurality of plurality of probes 140 immobilized on a top surface 101 of the substrate 100, and a capping layer 150 formed on a bottom surface of the substrate 100.

The substrate 100 may be a substrate including (e.g. is made of) a transparent material that allows visible light and/or UV light to pass through. For example, the substrate 100 may be a transparent substrate including glass, soda-lime glass, or quartz. When a glass substrate is used, it can be advantageously compatible with substrates that have been widely used for various known applications, including relatively thin slide substrates used in, for example, microscopic observation, relatively thick, large-screen liquid crystal display (LCD) panels, and so on. In other embodiments of the present invention, an opaque substrate may be employed.

Linkers 130 are formed on the top surface 101 of the substrate 100. The linkers 130 contain functional groups 135, each having a first end coupled to the top surface 101 of the substrate 100 and a second end coupled to the probes 140. When the probes 140 are DNA probes (e.g., oligo nucleotide probes), examples of the functional groups 135 that can be coupled to the probes 140 include hydroxyl groups, aldehyde groups, carboxyl groups, amino groups, amide groups, thiol groups, halo groups, and sulfonate groups.

When the substrate 100 includes glass, etc., the linkers 130 may include a silicon group capable of producing siloxane (Si—O) bonds with Si(OH) groups. Examples of materials used for the linkers 130, including the silicon group as well as the functional groups 135 that can be coupled to the probes 140 (hereinafter probes include monomers for probes), include N-(3-(triethoxysilyl)-propyl)-4-hydroxybutyramide, N,N-bis(hydroxyethyl) aminopropyl-triethoxysilane, acetoxypropyl-triethoxysilane, 3-Glycidoxy propyltrimethoxysilane), and silicon compounds disclosed in PCT application WO 00/21967, the content of which are hereby incorporated by reference.

As shown, the plurality of plurality of probes 140 are immobilized on the top surface 101 through the linkers 130, which will now be described in more detail. In the probe cell region I, the plurality of plurality of probes 140 are coupled to the second ends of the linkers 130. The linkers 130 may be formed on the entire top surface 101 of the substrate 100, regardless of whether they are in the probe cell region I or the non-probe cell region II. However, the probes 140 are selectively coupled to only the linkers 130 positioned in the probe cell region I of the substrate 100. In order to avoid synthesis noise or immobilization noise, the second ends of the linkers 130 positioned in the non-probe cell region II of the substrate 100 can have functional groups 135 inactively capped, as indicated by reference numeral 136. The functional groups 135 are capable of being coupled to the probes 140. Furthermore, according to some embodiments of the present invention, the linkers 130 may be selectively removed in the non-probe cell region II.

When the top surface 101 contains the functional groups 135 capable of being coupled to the probes 140, the linkers 130 may be omitted. In such embodiments, the functional groups 135 of the top surface 101 in the non-probe cell region II may be inactively capped or selectively removed, like the linkers 130.

The capping layer 150 is formed on the bottom surface 102 opposite to the top surface 101. In embodiments in which the bottom surface 102 contains functional groups 135 capable of being coupled to the probes 140, the capping layer 150 prevents undesired coupling, which may be caused by the functional groups 135, by preventing exposure of the functional groups 135. Accordingly, data noise can be reduced or avoided, thereby increasing the analysis reliability.

The capping layer 150 can include (e.g., is made of) a material without functional groups capable of being coupled to the linkers 130 or the probes 140, and examples thereof include a metallic film, a metal nitride film, a silicon nitride film, and the like. Examples of the metals include Ti, Ta, Cr, Al, Cu, Au, Ag, and alloys of these metals. In non-limiting exemplary embodiments, a Ti film or a TaN film is used as the capping layer 150.

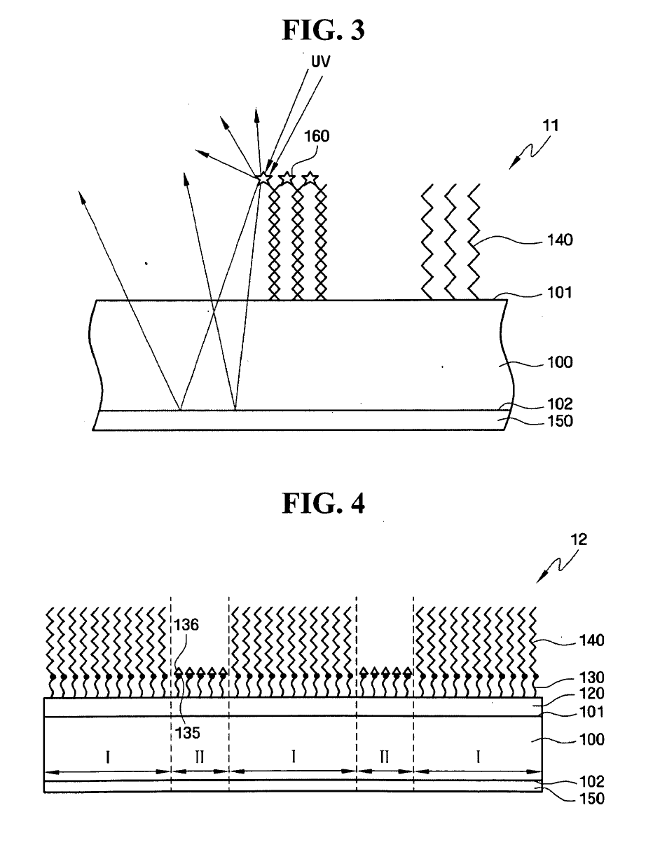

In some embodiments of the present invention, the capping layer 150 has a degree of reflectivity along with the aforementioned functions and can increase analysis efficiency during fluorescence detection, which will be described in greater detail with reference to FIG. 3.

FIG. 3 is a conceptual diagram of a fluorescence analysis in some embodiments of the present invention, in which linkers are not shown for brevity of explanation.

Referring to FIG. 3, it is assumed that the biochip 11 includes oligonucleotide probes as the probes 140. When single-stranded DNAs labeled with a fluorescent material 160 are provided as biological samples to the biochip 11, the single-stranded DNAs are bonded to the probes 140 having complementary base pairs by hybridization. If unreacted biological samples are removed, the single-stranded DNAs remain unreacted by being coupled to the probes. The single-stranded DNAs are labeled with the fluorescent material 160 only on particular probe cell regions I. If light of a predetermined wavelength, e.g., UV light, is irradiated onto the biochip 11, the labeled fluorescent material 160 can emit light having a particular wavelength. The emitted light can be collected by, for example, a scanner, and then analyzed to probe cells that have undergone hybridization, from which DNA base sequences of a target biological sample can be determined.

In order to deduce meaningful conclusions regarding DNA base sequences of a target biological sample, the amount of light collected from the fluorescent material 160 using a scanner should be sufficient. However, the scanner collecting emitted light is positioned on one side of the biochip 11, while using the fluorescent material 160 as a new spot light source, the light emitted from the fluorescent material 160 is scattered in all directions. Assuming that the scanner is positioned over the biochip 11, the maximum amount of light emitted from the fluorescent material 160 that can be then directly collected by the scanner 170 may not exceed 50%. Accordingly, it may not efficient to analyze data using only the directly collected light.

As a result, it can be advantageous to form the capping layer 150 using a material having reflectivity. That is to say, when the capping layer 150 is reflective, the light emitted downward (as shown in FIG. 3) from the fluorescent material 160 passes through the substrate, but is then reflected back by the capping layer 150 of the bottom surface 102 to travel upward again. The reflected light can be collected by the scanner and then used in data analysis. The more the amount of light is reflected, the higher the data analysis efficiency can become. In some embodiments, the reflectivity of the capping layer 150 is about 20% or higher.

The capping layer 150 can have a thickness that is related to the reliability of a capping ability and the efficacy of a reflecting ability. In some embodiments, for satisfactory capping and reflecting abilities, the capping layer 150 has a thickness in a range of about 1000 to about 3000 angstroms.

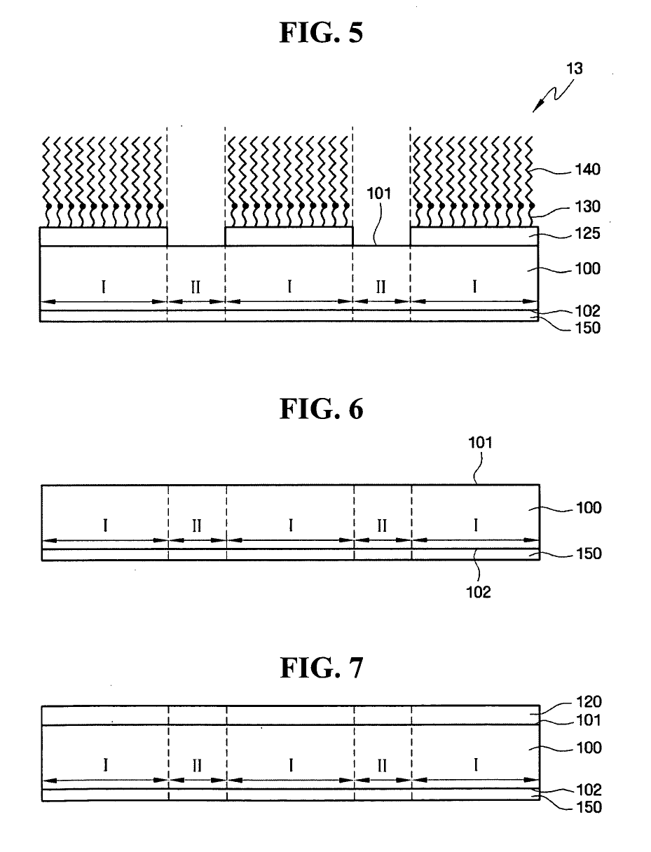

FIGS. 4 and 5 are sectional views of biochips according to other embodiments of the present invention.

Referring to FIGS. 2 and 4, a biochip 12 according to an embodiment of the present invention further includes an active layer 120 formed on the top surface 101. The active layer 120 may be formed on the entire top surface 101 of the substrate 100, regardless of whether it is in the probe cell region I or the non-probe cell region II. The linkers 130 are formed on the active layer 120.

When the top surface 101 is not coupled to the linkers 130 and/or the probes 140, or when there are negligible functional groups coupled to the linkers 130 and/or the probes 140, the active layer 120 may be advantageously provided. Further, the active layer 120 may include (e.g., be formed of) a material that is substantially stable against hydrolysis upon a hybridization assay, e.g., upon contact with a pH 6-9 phosphate or TRIS buffer. In some embodiments, the active layer 120 includes (e.g., is made of) a silicon oxide film such as a PE-TEOS film, a HDP oxide film, a P—SiH₄ oxide film or a thermal oxide film; a silicate such as hafnium silicate or zirconium silicate; a metal nitride film such as a silicon nitride film, a silicon oxynitride film, a hafnium oxynitride film or a zirconium oxynitride film; a metal oxide film such as ITO; a metal such as gold, silver, copper or palladium; a polyimide; a polyamine; or polymers such as polystyrene, polyacrylate or polyvinyl.

The active layer 120 may have a surface with a predetermined degree of roughness in order to ensure sufficient space for coupling with the linkers 130. For example, when the active layer 120 is formed of a thermal oxide film, it may have surface roughness of about 5 nm to about 100 nm.

The linkers 130 may include (e.g., be formed of) a material containing functional groups 135 each having a first end coupled to a top surface of the active layer 120 and a second end coupled to the probes 140. The material forming the linkers 130 may vary according to the material forming the active layer 120. When the active layer 120 includes (e.g., is made of), for example, a silicon oxide film, a silicate or a silicon oxynitride film, the linkers 130 may contain a silicon group capable of reacting with Si(OH) groups on the surface of the active layer 120 to produce siloxane (Si—O) bonds. Examples of materials are the same as described above with reference to FIG. 2. When the active layer 120 include (e.g., is made of) a metal oxide film, the functional group at one end of each of the linkers 130 coupled to the active layer 120 may include a metal alkoxide or metal carboxylate group. When the active layer 120 includes (e.g., is made of) a silicon nitride film, a silicon oxynitride film, a metallic oxynitride film, a polyimide, or a polyamine, the functional group at one end of each of the linkers 130 coupled to the active layer 120 may include anhydrides, acid chlorides, alkyl halides, or chlorocarbonates. When the active layer 120 includes a polymer, the functional group at one end of each of the linkers 130 coupled to the active layer 120 may include an acrylic, styryl, or vinyl group.

The plurality of probes 140 are immobilized on the active layer 120 of the top surface 101 via the linkers 130. That is to say, the plurality of plurality of probes 140 are coupled to the second ends of the linkers 130 in the probe cell region I to form probe cells. While the linkers 130 may be formed on the entire surface of the active layer 120, regardless of whether they are in the probe cell region I or the non-probe cell region II of the substrate 100, the probes 140 are selectively coupled to only the linkers 130 positioned in the probe cell region I of the substrate 100. In some embodiments, in order to avoid synthesis noise or immobilization noise, second ends of the linkers 130 positioned in the non-probe cell region II of the substrate 100 have functional groups 135 inactively capped, wherein the functional groups 135 are capable of being coupled to the probes 140. Furthermore, according to some modified embodiments of the present invention, the linkers 130 in the non-probe cell region II may be selectively removed.

When the surface of the active layer 120 contains functional groups capable of being coupled to the probes 140, the linkers 130 may be omitted. In such embodiments, the functional groups of the surface on the active layer 120 in the non-probe cell region II may be inactively capped or selectively removed, like the linkers 130.

The capping layer 150 is substantially the same as discussed above with reference to FIG. 2. Further, the capping layer 150 may contribute to selectively forming the active layer 120 only on the top surface 101 of the substrate 100. When the active layer 120 includes (e.g., is formed of), for example, a thermal oxide film, the capping layer 150 is formed on the bottom surface 102, followed by annealing. Then, the bottom surface 102 is covered by the capping layer 150 is not exposed, so that no thermal oxide film can be formed thereon, and a thermal oxide film is formed only on the top surface 101.

FIG. 5 is a sectional view of a biochip according to still another embodiment of the present invention.

Referring to FIG. 5, a biochip 13 according to another embodiment of the present invention further includes active patterns 125 formed on the top surface 101. The active patterns 125 are different from the active layer 120 shown in FIG. 4 in that they are selectively formed only on the probe cell regions I.

The linkers 130 are selectively formed only on the active patterns 125. The linkers 130 are not formed on the non-probe cell region II where the active patterns 125 are not positioned. As a result, the top surface 101 of the substrate may be directly exposed to the exterior environment in the non-probe cell region II. In some embodiments of the present invention, the top surface 101 of the non-probe cell region II may further include a coupling blocking film, a filler, and so on. Further details of structures of the top surface 101 in the non-probe cell region II are fully disclosed in Korea Patent Application Nos. 10-2006-0039713, and 10-2006-0039716, filed by the applicant of the present invention, the disclosures of which are incorporated herein in their entirety by reference. Even though the second ends of the linkers 130 are not inactively capped, unwanted coupling of the probes on the non-probe cell region II can be prevented in a more ensured manner by using the aforementioned structures, for example, as described for embodiments shown in FIG. 2 or 4. Accordingly, data noise can be further suppressed, thereby improving the analysis reliability.

Each of the active patterns 125 corresponds to the probe cell region I, and the active patterns 125 are physically discrete and spaced from each other. Therefore, from the formation of the active patterns 125 and the linkers 130, the respective probe cells of the biochip 13 can be independent of each other physically and chemically.

Materials forming the active patterns 125 can be substantially the same as those of the active layer 120 shown in FIG. 4. In addition, the other functional components can be substantially the same as those shown in FIG. 4, and a repeated explanation thereof will not be given.

Hereinafter, methods of fabricating biochips according to some exemplary embodiments of the present invention will be described by way of the biochip shown in FIG. 5.

FIGS. 6 through 9 are sectional views illustrating a method of fabricating biochips according to some embodiments of the present invention.

Referring to FIG. 6, a substrate 100 including a probe cell region I and a non-probe cell region II is provided. Next, a capping layer 150 is formed on a bottom surface 102 of the substrate 100. The capping layer 150 may be formed by a widely known deposition process including, for example, plasma enhanced chemical vapor deposition (PECVD), metal organic chemical vapor deposition (MOCVD), atomic layer deposition (ALD), plasma enhanced atomic layer deposition (PEALD), and the like.

Referring to FIG. 7, an active layer 120 is formed on a top surface 101 of the substrate 100. The active layer 120 can be formed by, for example, various deposition processes well-known in the art or a thermal oxidation process. When the thermal oxidation process is employed, the substrate 100 can be annealed at a temperature in a range of about 900 to about 1200° C. for about 3 to about 12 hours. Since the bottom surface 102 is protected by the capping layer 150, the active layer 120 formed of a thermal oxide film is selectively formed only on the top surface 101 of the substrate 100. The formed active layer 120 may have a surface roughness of about 5 nm to about 100 nm.

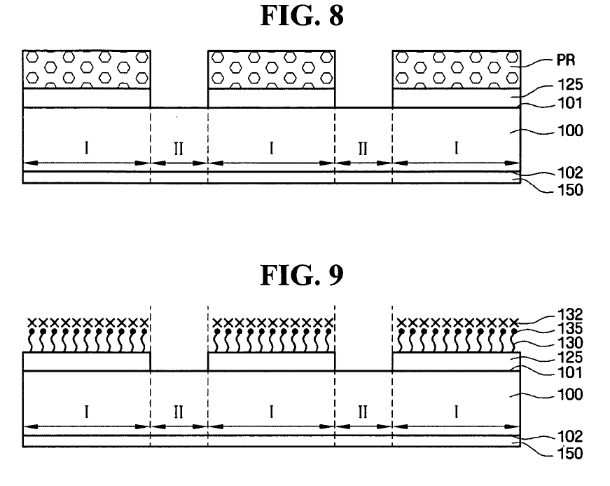

Referring to FIG. 8, photoresist patterns PR covering probe cell region I are formed on the active layer 120 and the active layer 120 is etched using the photoresist patterns PR as an etching mask to form the active patterns 125. Upon etching, the probe cell region I of the substrate 100 is covered by the active patterns 125, while the top surface 101 of the substrate 100 in the non-probe cell region I is exposed. Next, the photoresist patterns PR are removed.

Referring to FIG. 9, optionally, in order to modify the surface of the active patterns 125 so as to facilitate a reaction between the active patterns 125 and the linkers 130, the active patterns 125 are subjected to a surface treatment such as ozonolysis, acid treatment, or base treatment, using, for example, a Piranha solution (a mixture of sulfuric acid and hydrogen peroxide), a hydrofluoric acid solution, an ammonium hydroxide solution, or an O₂ plasma.

Subsequently, the linkers 130 are formed on the surface treated active patterns 125. wherein embodiments in which the probes 140 are formed using a photolithography process, which will be described below, photolabile groups 132 are attached to functional groups of the linkers 130. The linkers 130 are selectively formed only on the active patterns 125, and they are not formed on the exposed top surface 101 in the non-probe cell region II.

Referring back to FIG. 5, the probes 140 are formed on the linkers 130. When the functional groups 135 capable of coupling with the probes at second ends of the linkers 130 are protected by the photolabile groups 132, selective exposure of probe cell region I is performed to remove the photolabile groups 132, and the probes 140 are then coupled to the second ends of the linkers 130. For example, the coupling of the probes 140 may be performed by spotting onto completed probes, or synthesizing monomers for probes (e.g., nucleotide phosphoramidite monomers having functional groups protected by photolabile groups) by photolithography. After forming the probes 140, the biochip 13 shown in FIG. 5 is completed.

In some embodiments of the present invention in which it is not necessary or intended to increase reflectivity of a biochip, for example, where an analysis scheme other than fluorescence analysis may be employed, where a scanning method other than the use of the scanner may be employed, or where an opaque substrate may be used, removal of the capping layer 150 may be performed. The removing of the capping layer 150 can be carried out using, for example, a Piranha solution, or other cleaning solutions or a wet etching solution.

If the bottom surface 102 remains unreacted with the linkers 130, if the reactivity with the linkers 130 is insignificant, or if in the forming of the linkers 130, the linkers 130 are drastically prohibited from being provided to the bottom surface 102, then the capping layer 150, which has already contributed to preventing the active layer 120 from being formed on the bottom surface 102, may be removed in steps shown in FIG. 7 or 8. If the capping layer 150 is removed, the bottom surface 102 is exposed. Thus, even after the subsequent steps including coating, synthesis, exposure, scanning, and so on, it is not necessary to replace equipment due to a defect occurring in the equipment or a dimension error. In other embodiments of the present invention, removal of the capping layer 150 may be performed after the coupling of the probes 140.

To fabricate the biochip 11 shown in FIG. 2 or the biochip 12 shown in FIG. 4, some of the above-described steps may be modified. For example, to fabricate the biochip 11 shown in FIG. 2, the steps shown in FIGS. 7 and 8 are skipped. To fabricate the biochip 12 shown in FIG. 4, the steps shown in FIG. 8 are skipped.

More embodiments can be deduced from the description made with reference to FIGS. 6 through 9, together with FIG. 5, and a detailed explanation will not be given.

The present invention will be described in detail through the following experimental example. However, the experimental example is for illustrative purposes and other examples and applications can be readily envisioned by a person of ordinary skill in the art. Since a person skilled in the art can sufficiently understand the technical contents which are not described in the following experimental example, the description thereabout is omitted.

### EXPERIMENTAL EXAMPLE

A Ti film was deposited to a thickness of 2000 angstroms on a bottom surface of a glass substrate using a CVD process. Then, the Ti film was baked at 1000° C. for 5 hours to form a thermal oxide film having a thickness of about 5000 angstroms and a surface roughness of about 10 nm on a top surface of the glass substrate.

Then, a photoresist film was formed on the thermal oxide film to a thickness of about 3.0 μm using a spin-coating process and baked at 100° C. for 60 seconds. The photoresist film was exposed to light using a checkerboard-type, dark tone mask with a pattern of 1.0 μm in a 365 nm-wavelength projection exposure machine and developed with a 2.38% tetramethylammonium hydroxide (TMAH) solution to form checkerboard type photoresist patterns that expose the underlying film in the form of intersecting stripes. The thermal oxide film was etched using the photoresist patterns as an etching mask, thereby completing a plurality of active patterns corresponding to probe cell regions.

Next, the Ti film was removed from the bottom surface of the glass substrate using a piranha solution (7:3 concentrated H₂SO₄/H₂O₂) and functional groups on the active pattern surface were activated.

Next, the active patterns were spin-coated with bis(hydroxyethyl)aminopropyltriethoxysilane at 500 rpm for 30 seconds, and stabilized at room temperature for about 5 to 30 minutes. Then, the resultant product was treated with an acetonitrile solution containing NNPOC-tetraethyleneglycol and tetrazole (1:1) so that phosphoramidite protected with photolabile groups was coupled to the active patterns, and then acetyl-capped, which resulted in completion of protected linkers.

Next, the probe cell regions were exposed to light using a binary chrome mask, exposing desired active patterns in a 365 nm-wavelength projection exposure machine with an energy of 1000 mJ/cm² for one minute to deprotect terminating functional groups of the linkers. Then, the probe cell regions were treated with an acetonitrile solution containing amidite-activated nucleotide and tetrazole (1:1) to achieve coupling of the protected nucleotide monomers to the deprotected linkers, and then treated with a THF solution (acetic anhydride (Ac20)/pyridine (py)/methylimidazole=1:1:1) and a 0.02 M iodine-THF solution to perform capping and oxidation.

The above-described deprotection, coupling, capping, and oxidation processes were repeated to fabricate oligonucleotide probes having different sequences for each active pattern.

As described above, in biochips according to some embodiments of the present invention and fabrication methods thereof, unwanted coupling of linkers or probes to a bottom surface of a substrate can be prevented. Accordingly, data noise can be suppressed, thereby improving the analysis reliability. In addition, in biochips according to other embodiments of the present invention and fabrication methods thereof, an active layer or active patterns are selectively formed only on a top surface of the substrate. Furthermore, in biochips according to other embodiments of the present invention and fabrication methods thereof, use of a transparent substrate can increase analysis efficiency during fluorescence detection using a fluorescent material.

While the present invention has been particularly shown and described with reference to exemplary embodiments thereof, it will be understood by those of ordinary skill in the art that various changes in form and details may be made therein without departing from the spirit and scope of the present invention as defined by the following claims. It is therefore desired that the present embodiments be considered in all respects as illustrative and not restrictive, reference being made to the appended claims rather than the foregoing description to indicate the scope of the invention.

What is claimed is:

1. A biochip comprising:
a substrate;
a plurality of probes immobilized on a top surface of the substrate; and
a capping layer formed on a bottom surface of the substrate.

2. The biochip of claim 1, wherein the substrate is transparent.

3. The biochip of claim 2, wherein the capping layer has a reflectivity of about 20% or higher.

4. The biochip of claim 2, wherein the capping layer comprises a metallic film, a metal nitride film, or a silicon nitride film.

5. The biochip of claim 1, wherein the substrate includes a plurality of probe cell regions on which the plurality of probes are immobilized and non-probe cell isolation region on which the probes are not immobilized.

6. The biochip of claim 5, further comprising an active layer formed on the top surface of the substrate, wherein the plurality of probes are coupled to the active layer on the probe cell regions and not being coupled to the active layer on the non-probe cell region.

7. The biochip of claim 6, further comprising linkers formed on the active layer, wherein the linkers on the probe cell regions are coupled to the plurality of probes, and the linkers on the non-probe cell regions are not coupled to the probes.

8. The biochip of claim 1, further comprising active patterns selectively formed on the top surface of probe cell regions of the substrate and on which the plurality of probes are immobilized.

9. The biochip of claim 8, wherein the substrate comprises non-probe cell regions where the top surface of the substrate is exposed.

10. The biochip of claim 8, further comprising linkers formed on the active patterns and coupling the plurality of probes and the active patterns.

11. A method for fabricating a biochip comprising:
forming a capping layer on a bottom surface of a substrate; and
immobilizing a plurality of probes on a top surface of the substrate.

12. The method of claim 11, wherein the substrate is transparent.

13. The method of claim 12, wherein the capping layer has a reflectivity of about 20% or higher.

14. The method of claim 12, wherein the capping layer comprises a metallic film, a metal nitride film, or a silicon nitride film.

15. The method of claim 11, wherein immobilizing the plurality of probes comprises:
forming an active layer on the top surface of the substrate; and
coupling the plurality of probes on a predetermined region of the active layer.

16. The method of claim 15, wherein forming the active layer comprises subjecting the substrate to thermal oxidation.

17. The method of claim 15 further comprising forming linkers on the active layer after the forming of the active layer, wherein coupling the plurality of probes comprises coupling the plurality of probes on the predetermined region of the active layer through the linkers.

18. The method of claim 11, wherein immobilizing the plurality of probes comprises:
forming an active layer on the top surface of the substrate;
forming active patterns by patterning the active layer and coupling the plurality of probes on the active patterns.

19. The method of claim 18, further comprising removing the capping layer after forming the active patterns and before coupling the plurality of probes.

20. The method of claim 18 further comprising removing the capping layer after coupling the plurality of probes.
